(19) United States
(12) Patent Application Publication
(10) Pub. No.: US 2026/0038648 A1
(43) Pub. Date: Feb. 5, 2026

(54) METHOD, APPARATUS, DEVICE AND READABLE STORAGE MEDIUM FOR MOLECULE ENCODING

(71) Applicants: DOUYIN VISION CO., LTD., Beijing (CN); Lemon Inc., Grand Cayman (KY)

(72) Inventors: Tianze Zheng, Beijing (CN); Wen Yan, Los Angeles, CA (US); Chi Xu, Beijing (CN)

(21) Appl. No.: 19/286,732

(22) Filed: Jul. 31, 2025

(30) Foreign Application Priority Data
Aug. 2, 2024 (CN) 202411060313.6

(51) Int. Cl. G16C 20/90; G16C 20/20

(52) U.S. Cl. CPC G16C 20/90; G16C 20/20

(57) ABSTRACT

Embodiments in the disclosure provide a method and an apparatus for molecule encoding, a device, and a readable storage medium. The method includes determining, based on a molecular fragment structure of a target molecule, a root node corresponding to the molecular fragment structure; traversing the molecular fragment structure based on the root node to obtain traversal record information corresponding to the molecular fragment structure, the traversal record information corresponding to the molecular fragment structure indicating attribute information of each atom traversed along a traversal path corresponding to the traversal; and determining, by performing encoding on the traversal record information corresponding to the molecular fragment structure using a specified encoding rule, an encoding result corresponding to the molecular fragment structure, the encoding result including encoded representations of a plurality of atoms in the molecular fragment structure. In this way, the applicable scenarios of molecular encoding can be improved.

### CROSS REFERENCE

This application claims the benefit of Chinese Patent Application No. 202411060313.6, filed Aug. 2, 2024, entitled “METHOD, APPARATUS, DEVICE AND READABLE STORAGE MEDIUM FOR MOLECULE ENCODING”, the entirety of which are incorporated herein by reference.

### TECHNICAL FIELD

Example embodiments in the disclosure generally relate to the field of computer, and in particular, to a method and apparatus for molecule encoding, a device, and a readable storage medium.

### BACKGROUND

In the fields of chemistry and bioinformatics, the representation and analysis of molecular structures are important steps in studying molecular properties and behaviors. Common approaches include encoding molecular structures using encoding rules corresponding to SMiles Arbitrary Target Specification (SMARTS) to convert complex molecular structures into simple text-format strings, so as to facilitate computer processing and analysis.

However, existing encoding methods exhibit uncertainty. To match a target molecule, the textual string format needs to be parsed into a graph structure, which is then graph-matched with the target molecule. This not only increases computational complexity, but also leads to additional time consumption.

### SUMMARY

In a first aspect in the disclosure, a method for molecule encoding is provided. The method may include: determining, based on a molecular fragment structure of a target molecule, a root node corresponding to the molecular fragment structure; traversing the molecular fragment structure based on the root node to obtain traversal record information corresponding to the molecular fragment structure, the traversal record information corresponding to the molecular fragment structure indicating attribute information of each atom traversed along a traversal path corresponding to the traversal; and determining, by performing encoding on the traversal record information corresponding to the molecular fragment structure using a specified encoding rule, an encoding result corresponding to the molecular fragment structure, the encoding result comprising encoded representations of a plurality of atoms in the molecular fragment structure.

In a second aspect in the disclosure, an apparatus for molecule encoding is provided. The apparatus may include: a root node determining module configured to determine, based on a molecular fragment structure of a target molecule, a root node corresponding to the molecular fragment structure; a traversal record information determining module configured to traverse the molecular fragment structure based on the root node to obtain traversal record information corresponding to the molecular fragment structure, the traversal record information corresponding to the molecular fragment structure indicating attribute information of each atom traversed along a traversal path corresponding to the traversal; and an encoding result determining module configured to determine, by performing encoding on the traversal record information corresponding to the molecular fragment structure using a specified encoding rule, an encoding result corresponding to the molecular fragment structure, the encoding result comprising encoded representations of a plurality of atoms in the molecular fragment structure.

In a third aspect in the disclosure, an electronic device is provided. The device includes at least one processing unit and at least one memory, the at least one memory coupled to the at least one processing unit and storing instructions for execution by the at least one processing unit, the instructions, when executed by the at least one processing unit, causing the electronic device to perform the method according to the first aspect.

In a fourth aspect in the disclosure, a computer-readable storage medium is provided. The medium has a computer program stored thereon, the computer program, when executed by a processor, implementing the method according to the first aspect.

In a fifth aspect in the disclosure, a computer program product is provided. The computer program product includes computer-executable instructions, the computer-executable instructions, when executed by a processor, implementing the method according to the first aspect.

It should be understood that the content described in this section is not intended to limit key or important features of the embodiments in the disclosure, nor is it intended to limit the scope of the disclosure. Other features in the disclosure will become readily comprehensible through the following description.

### BRIEF DESCRIPTION OF THE DRAWINGS

The above and other features, advantages, and aspects of the embodiments in the disclosure become more apparent with reference to the following detailed description and in conjunction with the drawings. In the drawings, the same or similar reference numerals represent the same or similar elements, in which:

FIG. 1 shows a schematic diagram of an example environment in which embodiments in the disclosure can be implemented;

FIG. 2 shows a flowchart of a method for molecule encoding according to some embodiments in the disclosure;

FIG. 3 shows an example diagram of a target molecule according to some embodiments in the disclosure;

FIG. 4 shows an example diagram of a virtual root node determination principle according to some embodiments in the disclosure;

FIG. 5 shows a schematic diagram of a traversal result according to some embodiments in the disclosure;

FIG. 6 shows a schematic diagram of a traversal result according to other embodiments in the disclosure;

FIG. 7 shows a schematic diagram of a full traversal path according to some embodiments in the disclosure;

FIG. 8 shows a schematic structural block diagram of an apparatus for molecule encoding according to some embodiments in the disclosure; and

FIG. 9 shows a block diagram of an electronic device in which one or more embodiments in the disclosure can be implemented.

### DETAILED DESCRIPTION OF EMBODIMENTS

The embodiments in the disclosure are described in more detail below with reference to the drawings. Although some embodiments in the disclosure are shown in the drawings, it should be understood that the disclosure can be implemented in various forms and should not be construed as being limited to the embodiments set forth herein. Rather, these embodiments are provided for a thorough and complete understanding of the disclosure. It should be understood that the drawings and embodiments in the disclosure are only for example purposes, and are not intended to limit the protection scope of the disclosure.

In the description of the embodiments in the disclosure, the term “include/comprise” and similar terms should be understood as open-ended inclusion, that is, “include/comprise but not limited to”. The term “based on” should be understood as “based at least in part on”. The term “one embodiment” or “the embodiment” should be understood as “at least one embodiment”. The term “some embodiments” should be understood as “at least some embodiments”. The following may also include other explicit and implicit definitions.

In this document, unless expressly stated, performing a step “in response to A” does not mean that the step is performed immediately after “A”, but may include one or more intermediate steps.

It may be understood that data involved in the technical solutions in the disclosure (including, but not limited to, the data itself, the acquisition, use, storage or deletion of the data) should comply with the requirements of corresponding laws and regulations and related regulations.

It may be understood that before using the technical solutions disclosed in the embodiments in the disclosure, relevant users should be informed of the type, use scope, use scenario, etc. of information involved in the disclosure and authorization from the relevant users should be obtained through appropriate means in accordance with relevant laws and regulations, where the relevant users may include any type of rights holder, such as an individual, an enterprise, or a group.

For example, in response to receiving an active request from a user, prompt information is sent to the relevant user to explicitly prompt the relevant user that the operation requested to be performed will require acquisition and use of information of the relevant user, so that the relevant user can independently select whether to provide information to software or hardware such as an electronic device, an application, a server, or a storage medium that performs the operation of the technical solution in the disclosure according to the prompt information.

As an optional but non-restrictive embodiment, the manner of sending the prompt information to the relevant user in response to receiving the active request from the relevant user may be, for example, a pop-up window, and the prompt information may be presented in text in the pop-up window. In addition, the pop-up window may also carry a selection control for the user to select “agree” or “disagree” to provide information to the electronic device.

It may be understood that the above process of notifying and obtaining user authorization is only illustrative, and does not constitute a limitation on the embodiments in the disclosure. Other manners that satisfy relevant laws and regulations may also be applied to the embodiments in the disclosure.

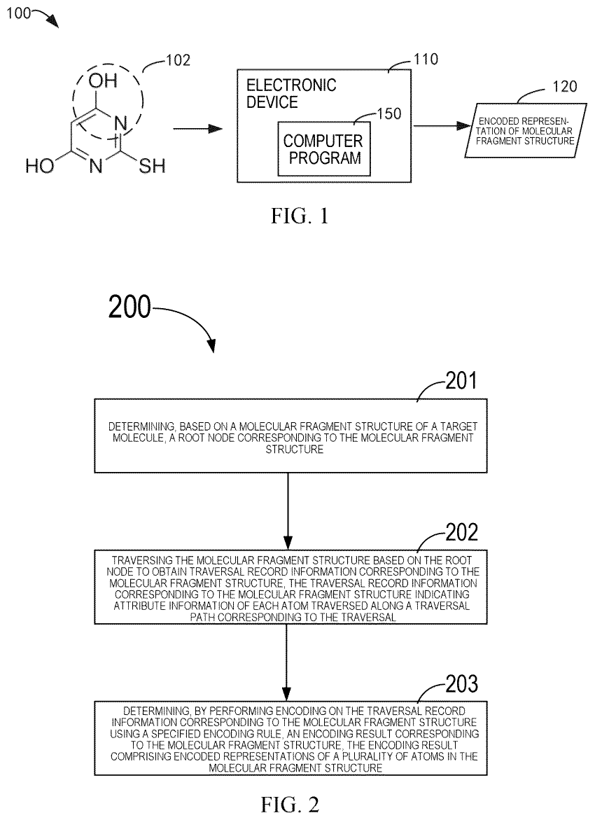

FIG. 1 shows a schematic diagram of an example environment 100 in which embodiments in the disclosure can be implemented. The environment 100 includes an electronic device 110. It is desirable that such an electronic device 110 is used to implement encoding of a molecular fragment structure 102 of a target molecule, so as to obtain an encoded representation 120 of the molecular fragment structure. To this end, in some embodiments, a computer program 150 may be installed and executed in the electronic device 110 to specify the encoding of the molecular fragment structure. The purpose of molecular fragment structure encoding is to accurately describe the local chemical environment of the molecule, which helps to improve the accuracy and efficiency of molecular simulation and energy computation.

For example, the electronic device 110 may include any computing system with computing power, such as various computing devices/systems, terminal devices, server-side devices, and the like. The terminal device may be any type of mobile terminal, stationary terminal or portable terminal, including a mobile phone, a desktop computer, a laptop computer, a notebook computer, a netbook computer, a tablet computer, a media computer, a multimedia tablet, a palmtop computer, a portable game terminal, a game device or any combination thereof, including accessories and peripherals of these devices or any combination thereof. The server-side device may be an independent physical server, a server cluster or distributed system composed of a plurality of physical servers, or a cloud server that provides basic cloud computing services such as cloud services, cloud databases, cloud computing, cloud functions, cloud storage, network services, cloud communications, middleware services, domain name services, security services, content delivery networks, and big data and artificial intelligence platforms. The server-side device may include, for example, a computing system/server, such as a mainframe, an edge computing node, a computing device in a cloud environment, and the like. It should be understood that the structure and function of each element in the environment 100 are described for example purposes only, without implying any limitation to the scope in the disclosure.

Currently, in the field of molecular structure encoding, conventional methods typically rely on manually defined rules, which results in uncertainty in the generated encoding and can easily lead to inconsistencies and errors. For complex molecular structures, the process of manually defining rules is labor-intensive and prone to mistakes. During the encoding of ring-containing molecules, traditional methods have difficulty avoiding duplicate encoding, leading to non-unique encoding results and reduced accuracy in molecular matching. In addition, parsing the encoding into a graph structure and then performing graph matching with the molecule is a complex task, characterized by high computational complexity and low efficiency, making it difficult to be applied rapidly in large-scale molecular libraries.

For example, a molecule may be represented by a graph, that is, G=(V,E), where V may represent a set of atoms in the target molecule, and E may represent a set of chemical bonds between pairs of atoms. vi may represent an i-th atom in the target molecule, and the atom may include features such as an element type, a connection count, aromaticity, a smallest ring to which the atom belongs, and a ring connection count, where vi EV. E may represent a chemical bond connecting an i-th atom vi and a j-th atom vj, and the chemical bond has features such as a bond order (single, double, triple or aromatic bond) or whether it is in a ring. A molecular fragment structure may be represented as a representation of an induced subgraph, that is, G′=G [V′]=(V′,E′), ∀vi′∈V′, ∀eij′∈E′. G′ may represent the induced subgraph. V′ may represent a set of nodes in the induced subgraph G′, that is, atoms in a local region. E′ may represent a set of edges in the induced subgraph G′, that is, chemical bonds in the local region. vi′ may be an i-th atom in the induced subgraph G′, and may have corresponding partial features. eij′ may be a chemical bond connecting an atom vi′ and an atom vj′ in the induced subgraph G′, and may have corresponding partial features.

In a chemical environment, the representation of a fully connected subgraph means that all atoms and bonds between them are interconnected, with no isolated atoms or disconnected bonds. Based on this, the chemical environment may be defined as: f(G′)=(fv(V′),fe(E′)), fv and fe acting on atoms and bonds respectively, and retaining at least partial features of the atoms and bonds. fv(V′) may form a feature set corresponding to partial features of all atoms in the subgraph, and fe(E′) may form a feature set corresponding to partial features of all chemical bonds in the subgraph.

f(G′) may be understood as a set of different subgraphs. Considering that G′ must meet the topological relationship and the features of atoms and bonds required by f(G′), it follows that G′∈f(G′). Meanwhile, since fv and fe only retain partial features of atoms and bonds, for any subgraph G″ that differs from G′ only in retained, it is also holds that G″∈f(G′).

In the process of molecule analysis, there is often a process of molecule recognition. For example, for a given molecule, graph matching needs to be performed based on a set of different subgraphs f(G′). For an electronic device, the complexity of graph matching is relatively high.

The embodiments in the disclosure propose a solution for molecule encoding. According to the solution, a root node corresponding to a molecular fragment structure is determined based on a molecular fragment structure of a target molecule. The molecular fragment structure is traversed based on the root node to obtain traversal record information corresponding to the molecular fragment structure, the traversal record information corresponding to the molecular fragment structure indicating attribute information of each atom traversed along a traversal path corresponding to the traversal. An encoding result corresponding to the molecular fragment structure is determined by performing encoding on the traversal record information corresponding to the molecular fragment structure using a specified encoding rule, the encoding result including encoded representations of a plurality of atoms in the molecular fragment structure.

Accordingly, by traversing the molecular fragment structure and performing encoding on traversal record information using a specified encoding rule, this automated encoding method eliminates errors introduced by manual rule writing and improves consistency and accuracy. The traversal record information can indicate the attribute information of each atom, ensuring that the encoding result accurately reflects the local chemical environment of the molecule. By determining a root node and traversing the molecular fragment structure starting from the root node, uniquely determined traversal record information is generated. The traversal record information records the attribute information of each atom along the traversal path. Even in the case where the molecular fragment structure contains a ring, the traversal path can still be uniquely determined. This avoids duplicate encoding and ensures the uniqueness and accuracy of the encoding result. In addition, the encoding result can be directly generated based on the traversal record information, avoiding the complex processes of graph parsing and matching, and enabling matching to be performed based on the encoded representation. This reduces computational complexity and improves efficiency.

The embodiments in the disclosure are described in detail below with reference to the drawings. FIG. 2 shows a flowchart of a process 200 of molecule encoding according to some embodiments in the disclosure. The process 200 may be implemented at the electronic device 110. For ease of discussion, the process 200 is described below with reference to FIG. 1.

As shown in FIG. 2, at block 201, the electronic device 110 determines, based on a molecular fragment structure of a target molecule, a root node corresponding to the molecular fragment structure. The molecular fragment structure may include one atom in the target molecule, a bond or a bond angle connecting at least two atoms, a carbon-carbon bond in a benzene ring, and the like.

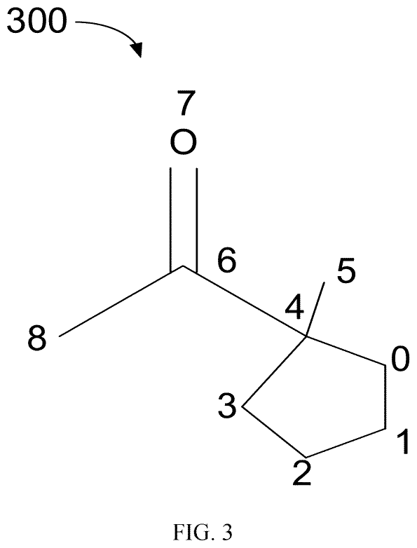

For each molecular fragment structure, a root node corresponding to the molecular fragment structure may be determined. FIG. 3 shows a schematic diagram of a molecular fragment structure 300 according to some embodiments in the disclosure. For example, if the molecular fragment structure corresponds to a bond angle formed by atoms labeled 4, 6, 7, and 8 in FIG. 3, an atom labeled 6 (the central atom of the bond angle) may be used as the root node. It should be noted that, in the presentation of the molecular fragment structures involved in the present disclosure and during the encoding process, hydrogen atoms are omitted. The purpose of such omission is to reduce the encoding size.

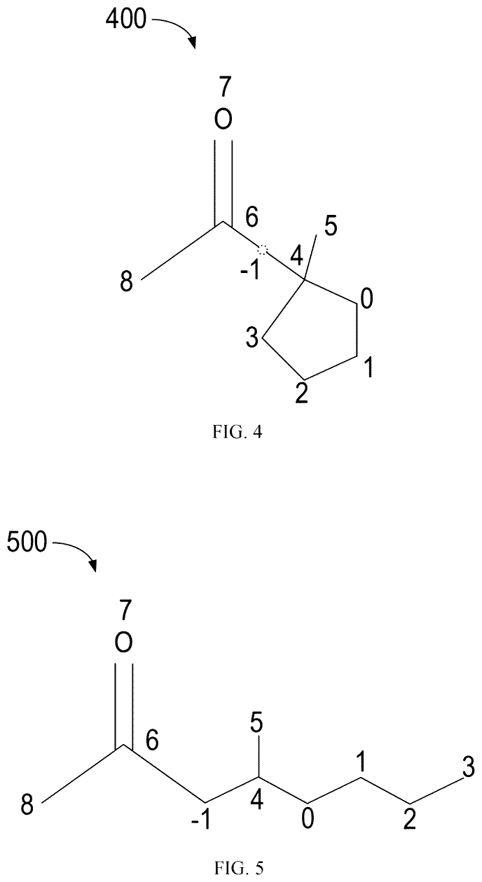

If the molecular fragment structure corresponds to a bond formed by atoms labeled 4 and 6 in FIG. 3, a virtual root node may be created between the two central atoms (the atoms labeled 4 and 6) of the molecular fragment structure. The bond between the two atoms (the atoms labeled 4 and 6) is broken, and the two atoms (the atoms labeled 4 and 6) are respectively connected to the created virtual root node. FIG. 4 shows a schematic diagram of a creation principle 400 for creating a virtual root node according to some embodiments in the disclosure. In FIG. 4, a node labeled −1 corresponds to the created virtual root node. After the virtual root node is created, it is necessary that the originally connected bond between the two atoms (labeled 4 and 6) is disconnected, and the two atoms (labeled 4 and 6) are respectively connected to the created virtual root node.

At block 202, the electronic device 110 traverses the molecular fragment structure based on the root node to obtain traversal record information corresponding to the molecular fragment structure, the traversal record information corresponding to the molecular fragment structure indicating attribute information of each atom traversed along a traversal path corresponding to the traversal.

The molecular fragment structure is traversed with the root node as the traversal starting point. FIG. 5 shows a schematic diagram of a traversal result 500 according to some embodiments in the disclosure for the molecular fragment structure corresponding to a bond formed by atoms corresponding to reference numerals 4 and 6 in FIG. 3. The traversal result may be that the molecular fragment structure is expanded into a tree structure. The virtual root node labeled −1 may be used as the starting point for traversing the molecular fragment structure.

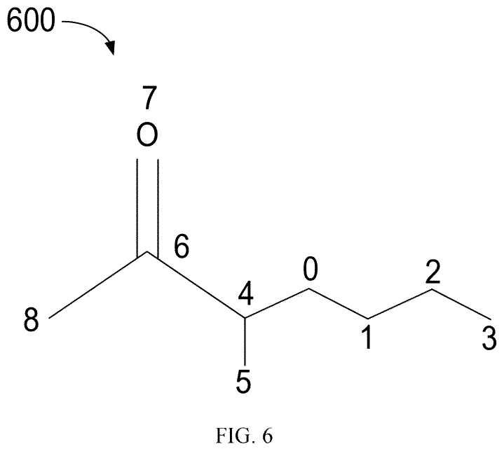

For a molecular fragment structure corresponding to a bond angle formed by the atoms labeled 4, 6, 7, and 8 in FIG. 3, FIG. 6 illustrates a schematic diagram 600 of a traversal result according to some embodiments in the disclosure. The atom labeled 6, corresponding to the root node, may be used as the starting point for traversing the molecular fragment structure.

Starting from the root node, the atoms included in the molecular fragment structure are traversed, and traversal record information is generated. The traversal record information may include attribute information of the atoms along the traversal path. The attribute information may indicate feature information of the corresponding atom. If the molecular fragment structure corresponds to a bond formed by the atoms labeled 4 and 6 in FIG. 3, the feature information of the atoms labeled 4 and 6 may be full information. For example, the attribute information may indicate features such as element type, aromaticity, connection count, ring connection count, and smallest ring to which the atoms labeled 4 and 6 belong.

For atoms outside the molecular fragment structure shown in FIG. 3 (i.e., atoms other than the atoms labeled 4 and 6), the attribute information may indicate only basic information. For example, only the element type and connection count may be retained. Based on this, various types of molecular fragment structures can be represented through traversal record information. The specific process of traversing the molecular fragment structure will be described in detail later.

At block 203, the electronic device 110 determines, by performing encoding on the traversal record information corresponding to the molecular fragment structure using a specified encoding rule, an encoding result corresponding to the molecular fragment structure, the encoding result including encoded representations of a plurality of atoms in the molecular fragment structure.

After the traversal record information corresponding to the molecular fragment structure is determined, the specified encoding rule may be used to perform encoding on the traversal record information to obtain the encoding result. Since the traversal record information corresponds to attribute information of atoms in the molecular fragment structure, such as element type, aromaticity, unber of connections, and the like, the encoding result includes encoded representations of the plurality of atoms in the molecular fragment structure. Moreover, the encoded representations may indicate various features of the atoms.

The specified encoding rule may include an encoding rule corresponding to SMARTS, which is used to encode the molecular fragment structure of the target molecule to obtain a string that describes the local chemical environment. In the encoding rule corresponding to SMARTS, different atom features may be expressed through different string symbols. For example, the description of atoms may include: element type (corresponding to the symbol “#”), number of connections (corresponding to “X”), aromaticity (corresponding to “A/a”), smallest ring to which the atoms belong (corresponding to “r”), ring connection count (corresponding to “x”), and the like. The description of chemical bonds may include: the symbol “-” for single bonds, “=” for double bonds, “#” for triple bonds, and “:” for aromatic bonds. The symbols “@” and “!@” may respectively represent ring bonds and non-ring bonds, among others. For the SMARTS-based encoding rule, ambiguous features may be removed. For example, if a feature is described by a numerical range, it may be considered ambiguous and therefore omitted. As an example, with respect to rings, if the encoding rule includes a feature described as smaller or larger than an N-membered ring (where N is a positive integer), such a feature is described by a numerical range and may be discarded. In contrast, specific values such as three-membered or five-membered rings may be retained.

In addition, the specified encoding rule may also include an encoding rule corresponding to Simplified Molecular Input Line Entry System (SMILES), an encoding rule corresponding to International Chemical Identifier (InChI), and the like.

Through the above process, encoded representations may be automatically generated based on the molecular fragment structure of the target molecule and the specified encoding rule, allowing for the processing of a greater variety of atom types and more complex molecular structures, thereby meeting the demands of large-scale datasets. By traversing the molecular fragment structure and recording the attribute information of each atom along the traversal path, complex or specific chemical structures may be more accurately identified.

The electronic device 110 may determine the traversal record information in various embodiments. In an embodiment, the determination of the traversal record information by the electronic device 110 may include: traversing, based on a first traversal order, the atoms in the molecular fragment structure using the root node as a first node to be traversed; recording, for each traversed atom, a traversal mark of the atom, the traversal mark of each atom used to indicate at least a traversal path corresponding to the atom.

Obtaining, based on the traversal mark of each atom, traversal record information corresponding to each atom; and generating, based on the traversal record information corresponding to each atom, the traversal record information corresponding to the molecular fragment structure.

The first traversal order may be a depth-first traversal order with the root node as the first node to be traversed. The depth-first traversal order may refer to starting from the root node, going along the traversal path until reaching the deepest level where it can no longer move forward, and then backtracking to the root node to continue exploring the next branch.

During the process of traversing atoms in the molecular fragment structure in a first traversal order, the electronic device 110 accesses each atom and sequentially records a traversal marker for the atom. By way of example, the traversal marker may indicate the traversal path corresponding to the atom, the identifier of the atom, and so on. In conjunction with the example shown in FIG. 4, using the atom's label in the figure as the identifier, the traversal process is as follows. The first traversal path: the traversal starts with the virtual root node labeled −1, followed by the node labeled 6. A branch occurs at the node labeled 6, and two branches are traversed in sequence, i.e., the node labeled 7 and the node labeled 8 (until there are no deeper nodes to traverse). By way of example, for the node labeled 6, its traversal marker may indicate: label 6, the first traversal path, and connection to the node labeled −1. For the node labeled 7, its traversal marker may indicate: label 7, one of the branches of the first traversal path, and connection to the node labeled 6. For the node labeled 8, its traversal marker may indicate: label 8, another branch of the first traversal path, and connection to the node labeled 6. Since the nodes labeled 7 and 8 cannot be further traversed to deeper levels, the first traversal path ends.

The traversal then continues with a second traversal path. The second traversal path begins with the virtual root node labeled −1, followed by the node labeled 4. A branch occurs at the node labeled 4, and the branches are traversed in sequence. The first branch includes the node labeled 5. The second branch sequentially includes the nodes labeled 0, 1, 2, and 3. The third branch sequentially includes the nodes labeled 3, 2, 1, and 0. As a result, the second traversal path ends. Similarly, based on the second traversal path, a traversal marker may be obtained for each node.

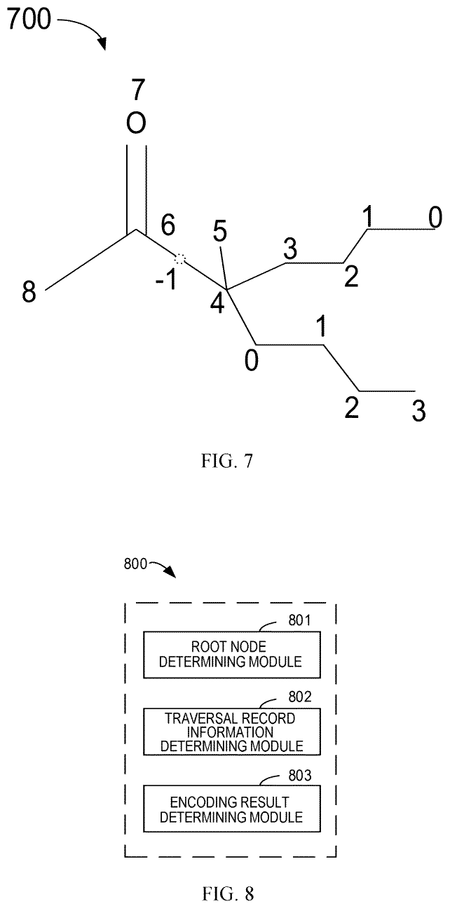

The first traversal path and the second traversal path together correspond to a full traversal path of the tree structure. FIG. 7 illustrates a schematic diagram 700 of a full traversal path of the molecular fragment structure corresponding to FIG. 4, according to some embodiments in the disclosure. After all traversals are completed, a traversal marker may be obtained for each atom corresponding to each traversal path. Based on the traversal markers of the respective atoms, attribute information such as chemical element type, connection count, ring marker, and so on, for each atom may be determined. By aggregating the traversal record information corresponding to each atom, traversal record information corresponding to the molecular fragment structure may be obtained.

After traversing the molecular fragment structure to obtain the traversal marks of the respective atoms, the traversal marks may be processed to determine the traversal record information of the atoms. Specifically, for the traversal mark of each traversed atom, in response to the traversal mark indicating that the atom is recorded in an existing traversal path, the atom is determined as a ring atom. The traversal record information corresponding to the atom is generated, the traversal record information indicating at least the chemical element type, the ring atom mark, the ring connection count, the bond feature, and the like corresponding to the atom.

Continuing with the example shown in FIG. 7, the second traversal path begins with the virtual root node labeled −1, followed by the node labeled 4. At the node labeled 4, three branches occur and are traversed in sequence. The first branch includes the node labeled 5. The second branch sequentially includes the nodes labeled 0, 1, 2, and 3. The third branch sequentially includes the nodes labeled 3, 2, 1, and 0. During the traversal process, the electronic device 110 records the traversal path taken to reach each node. When a newly accessed node already exists in the recorded traversal path, traversal (of the current branch or current path) stops. In addition, bond features between the ring-forming node and its connected nodes, as well as the order of appearance of the ring-closing node in the path, are recorded as part of the ring-forming marker.

For example, with respect to the second branch, the first node is the node labeled 4, followed by the nodes labeled 0, 1, 2, and 3 in sequence. After completing the traversal of the node labeled 3, the traversal continues to the node labeled 4. Since the node labeled 4 already exists in the recorded traversal path, traversal of the second branch is stopped. Based on this, it may be determined that the nodes labeled 4, 0, 1, 2, and 3 form a ring structure with a ring connection count of 5. In addition, based on the bond-related information obtained during the traversal process, it may be determined that the connection count for each atom is 1.

Similarly, with respect to the third branch, the first node is the node labeled 4, followed by the nodes labeled 3, 2, 1, and 0 in sequence. After completing the traversal of the node labeled 0, the traversal continues to the node labeled 4. Since the node labeled 4 already exists in the recorded path, traversal of the third branch is stopped. Based on this, it may also be determined that the nodes labeled 4, 3, 2, 1, and 0 form a ring structure with a ring connection count of 5. In addition, based on the bond-related information obtained during the traversal process, it may be determined that the connection count for each atom is 1.

The beneficial effect of the above process is that the molecular fragment structure can be expanded into a tree structure. Each node in the tree structure may represent an atom or a group of atoms, and each edge may represent a chemical bond. The tree structure is unique and well-defined. As a result, the accuracy and completeness of chemical structure identification can be improved.

In addition, to satisfy the uniqueness of the tree structure, the electronic device 110 may further perform sorting on encoded representations of a plurality of atoms. Specifically, the encoded representations of a plurality of atoms may be in the form of strings, and determining the encoding result corresponding to the molecular fragment structure may include: performing encoding on the traversal record information corresponding to the molecular fragment structure using the specified encoding rule to obtain an initial encoding result, the initial encoding result including initial encoded representations of a plurality of atoms in the molecular fragment structure; and sorting the initial encoded representations of the plurality of atoms according to a lexicographic order to obtain the encoding result corresponding to the molecular fragment structure.

By performing encoding on the traversal record information corresponding to the molecular fragment structure using the specified encoding rule, an initial encoding result may be obtained. As previously described, the traversal record information is derived from the traversal record information of each atom. Accordingly, the initial encoding result may indicate the initial encoded representations of a plurality of atoms in the molecular fragment structure. By way of example, each atom's encoded representation may be in the form of a string.

Based on the string-form encoded representations, the initial encoded representations of a plurality of atoms may be sorted to obtain the encoding result corresponding to the molecular fragment structure. As previously described, the molecular fragment structure can be expanded into a tree structure through traversal. Based on this, a first round of sorting may be performed on the tree structure, namely, starting from the root node, recursively performing encoding on the traversal record information of each child node to obtain the initial encoded representations of a plurality of atoms.

The sorting of the initial encoded representations of a plurality of atoms may be based on lexicographic order. The purpose of sorting is to ensure consistency and comparability of the initial encoded representations. For example, sorting may ensure that identical molecular fragment structures generate the same encoding result. Furthermore, sorting enables direct comparison of different molecular representations. Even if two molecules have similar topological structures, their differing chemical environments may be clearly distinguished through the sorted representations. By way of example, taking the sorted result of the encoding performed on the full traversal path shown in FIG. 7, the initial encoded representations of a plurality of atoms may be: “(−!@[#6AX3x0r0:](˜[#6X4])(˜[#8X1]))(−!@[#6AX4x2r5:](˜[#6X4])(˜[#6X4](˜[#6X4](˜[#6X 4](˜[#6X4]˜1))))(˜[#6X4](˜[#6X4](˜[#6X4](˜[#6X4]˜1”.

A partial explanation of the initial encoded representation is as follows. In the encoded string (−!@[#6AX3x0r0:](˜[#6X4])(˜[#8X1])), the symbol “−!@” may indicate a non-ring single bond. The string [#6AX3x0r0:] may represent a carbon atom (C), with a connection count of 3 (X3), not in a ring (r0). The string (˜[#6X4]) may represent a connection to a tetravalent carbon atom (C), via any bond ( ) The string (˜[#8X1]) may represent a connection to a monovalent oxygen atom (O), also via any bond ( ) Other encoded representations may refer to the corresponding SMARTS encoding rules and are not elaborated here.

Furthermore, for ring-forming atoms encountered during traversal, as such atoms are traversed repeatedly, the repeated traversals need to be deduplicated. The deduplication process performed by the electronic device 110 may include: performing duplicate checking on the encoded representations of the plurality of atoms based on a second traversal order to obtain a duplicate checking result; in response to the duplicate checking result indicating that there are repeated encoded atoms in the encoded representations of the plurality of atoms, performing deduplication on the repeated encoded atoms to obtain a deduplicated encoding result, the deduplicated encoding result including the encoded representations of the plurality of atoms retained after the deduplication; and sorting the encoded representations of the plurality of atoms after the deduplication according to a lexicographic order to obtain the encoding result corresponding to the molecular fragment structure.

As previously described, the first traversal order may be a depth-first traversal order starting from the root node. Correspondingly, the second traversal order may be a breadth-first traversal order starting from the root node.

In conjunction with FIG. 7, based on the breadth-first traversal order, consider the encoded representations of a plurality of atoms in the molecular fragment structure. Suppose that the second path is first traversed, and its second branch includes the nodes labeled 3, 2, 1, and 0. Thereafter, the third branch of the second path is traversed, and it includes the nodes labeled 0, 1, 2, and 3. In response to encountering the node labeled 0 in the third branch, and the fact that this node has already been recorded during the traversal of the second branch, the node labeled 0 can be identified as a duplicate encoded atom. Similarly, in response to encountering the nodes labeled 1 through 3 in the third branch, these nodes can also be sequentially identified as duplicate encoded atoms. Accordingly, the encoded representations corresponding to the duplicate encoded atoms may be deduplicated to obtain a deduplicated encoding result. After deduplication is applied to the encoded result of the tree structure shown in FIG. 7, the resulting structure corresponds to the molecular structure shown in FIG. 5.

Since the deduplication operation removes duplicate encoded atoms, this may lead to changes in the connection count, ring-forming markers, and other attribute information of the remaining atoms. Therefore, it is necessary to update the traversal record information corresponding to the deduplicated atoms. Based on the updated traversal record information, encoding is performed to obtain the deduplicated encoding result. After the deduplicated encoding result is obtained, the encoded representations of a plurality of retained atoms in the result may still be sorted in lexicographic order to determine the encoding result corresponding to the molecular fragment structure. By way of example, the molecular structure shown in FIG. 5 is the deduplicated molecular structure, and the sorted deduplicated encoding result obtained based on it may be:

“(−!@[#6AX3x0r0:](˜[#6X4])(˜[#8X1])) (−!@[#6AX4x2r5:](˜[#6X4])(˜[#6X4] (˜[#6X4]˜1))(˜[#6X4] (˜[#6X4]˜1)))”. The symbol “˜1” indicates the ring-forming positions (appearing in pairs). By comparing the initial encoded representations of a plurality of atoms, it can be seen that the number of strings in the deduplicated encoded representation has been reduced.

The re-sorting ensures that changes in atomic attribute information during the deduplication process do not result in non-unique encoding results. This guarantees the uniqueness and determinacy of the encoding result for the molecular fragment structure.

To ensure the uniqueness of the encoding result corresponding to the molecular fragment structure, in some embodiments, the electronic device 110 may assign a respective number to each atom among a plurality of retained atoms after deduplication. Based on the assigned numbers, the traversal record information corresponding to the molecular fragment structure is updated. Encoding is then performed on the updated traversal record information to obtain a deduplicated encoding result, which includes encoded representations of each of the plurality of retained atoms after deduplication.

For the deduplicated encoding result, number assignment may also be performed. Taking FIG. 4 as an example, the molecular fragment structure corresponds to the bond formed by the atoms labeled 4 and 6 in FIG. 4. By way of example, the retained atoms after deduplication include the atoms labeled 4 and 6. Based on this, respective numbers may be assigned to the atoms labeled 4 and 6. The numbers are typically represented using digits or letters.

Based on the assigned numbers, the traversal record information corresponding to the molecular fragment structure is updated. Since the traversal record information is derived from the traversal markers of individual atoms, the updated traversal record information corresponding to the molecular fragment structure includes the assigned numbers.

Encoding is then performed on the traversal record information corresponding to the molecular fragment structure to obtain the deduplicated encoding result. In other words, the deduplicated encoding result includes an encoded representation of each of the plurality of retained atoms after deduplication, and the encoded representation of each atom may indicate the number assigned to that atom.

There may be a plurality of possible ways to assign numbers, and all number assignment methods need to be traversed. Taking FIG. 4 as an example, number assignment method 1 may assign number 1 to the atom labeled 4 and number 2 to the atom labeled 6. By way of example, for each number assignment method, a corresponding deduplicated encoding result will be generated. The encoded representation including number 1 may be: “[#6AX3x0r0:1] (˜[#6X4])(˜[#8X1])−!@[#6AX4x2r5:2] (˜[#6X4])(˜[#6X4]˜[#6X4]˜1)˜[#6X4] ˜[#6X4]˜1”.

In addition, number assignment method 2 may assign number 2 to the atom labeled 4 and number 1 to the atom labeled 6. By way of example, the encoded representation including number 2 may be:

“[#6AX3x0r0:2] (˜[#6X4])(˜[#8X1])−!@[#6AX4x2r5:1] (˜[#6X4])(˜[#6X4]˜[#6X4]˜1)˜[#6X4] ˜[#6X4]˜1”.

By traversing all possible number assignment methods, a corresponding number of deduplicated encoding results may be obtained. In cases where the encoding result is a string, all of the deduplicated encoding results may be compared in lexicographic order. Based on the comparison result, either the lexicographically smallest or the largest may be selected as the encoding result corresponding to the molecular fragment structure. A unified selection criterion (smallest or largest) may be adopted for the encoding process. By selecting the lexicographically smallest result, the encoded representation including number 1 may be selected as the final result.

Through the above process, to generate a unique and deterministic encoding result corresponding to a molecular fragment structure, different numbering schemes need to be considered, and the smallest or largest one among them should be selected. This approach can handle various types of chemical structures while ensuring that the encoding result is both unique and deterministic. For example, for bonds, bond angles, and regular dihedral angles (torsion angles between four atoms), there are usually 2 possible number assignment methods. For irregular dihedral angles, there are typically 6 number assignment methods. By utilizing assigned numbers, it is ensured that the encoding results corresponding to various complex molecular fragment structures remain unique and deterministic.

In some embodiments, the electronic device 110, in response to the molecular fragment structure including a first symmetric structure, may determine that the first symmetric structure indicates a structure centered on a central atom. The central atom of the first symmetric structure is used as the root node.

The first symmetric structure may indicate the presence of a symmetric structure in the molecular fragment that is centered on a central atom. Such a symmetric structure is typically characterized by other atoms or atom groups symmetrically distributed around the central atom. For example, the structure formed by the carbon atoms and their attached hydrogen atoms in a benzene ring is a typical symmetric structure. Upon identifying the first symmetric structure, the central atom of the structure is located. The central atom is the core around which all other atoms in the symmetric structure are symmetrically arranged. For instance, in a benzene ring, any carbon atom can be considered a central atom, as each one occupies an equivalent structural position. By adopting this method, the selected root node during encoding of the molecular fragment structure can maintain both uniqueness and symmetry, thereby simplifying the subsequent traversal and encoding process.

In some embodiments, the electronic device 110, in response to the molecular fragment structure including a second symmetric structure, may determine that the second symmetric structure indicates a structure centered on a bond. A virtual root node is set between the two atoms connected by the bond, and the two atoms are respectively connected to the virtual root node. On this basis, determining the encoding result corresponding to the molecular fragment structure comprises: removing the encoded representation corresponding to the virtual root node to obtain encoded representations of the remaining atoms; and determining the encoding result corresponding to the molecular fragment structure based on sorting the encoded representations of the remaining atoms.

The second symmetric structure may indicate a bond-centered symmetric structure, which typically refers to symmetry exhibited by both ends of a bond and their connected components. To ensure that such a structure is encoded uniquely and deterministically, a virtual root node may be introduced. The virtual root node is an artificial node (not a real node) used to assist in building the tree structure of the molecular fragment. The virtual root node is connected to the two atoms at both ends of the bond, forming a new tree structure. After traversal is completed using the virtual root node and the corresponding encoded representation of the molecular fragment structure is obtained, the encoded representation of the virtual root node is removed, as it is included in the encoding result.

After the encoded representation corresponding to the virtual root node is removed, a new root node may be selected based on a specified priority from among the nodes nearest to the deleted virtual root node. The specified priority may be determined based on the first traversal order, the second traversal order, or lexicographic order. After the new root node is determined, traversal of the atoms in the molecular fragment structure is performed starting from the new root node based on the first traversal order. Traversal record information corresponding to the molecular fragment structure is then generated based on the traversal record information of the respective atoms, and encoding is performed on the traversal record information to obtain the encoding result corresponding to the molecular fragment structure.

By way of example, in the preceding example, the sorted deduplicated encoding result of the deduplicated molecular structure may be: #6X4](˜[#6X4]˜1)))”. In the encoded representation containing the virtual root node, the two main parts are separated by the virtual root node. The encoded representation of the first part is: “(−!@[#6AX3x0r0:](˜[#6X4])(˜[#8X1]))”.

The encoded representation of the second part is: “(−!@[#6AX4x2r5:](˜[#6X4])(˜[#6X4] (˜[#6X4]˜1))(˜[#6X4] (˜[#6X4]˜1)))”.

After the virtual root node is removed, the encoding result corresponding to the molecular fragment structure may be: “[#6AX3x0r0:](˜[#6X4])(˜[#8X1])−!@[#6AX4x2r5:](˜[#6X4])(˜[#6X4]˜[#6X4]˜1)˜[#6X4]˜[#6X4]˜1”. In this way, in the process of number assignment, the encoding result corresponding to the molecular fragment structure after removal of the virtual root node may be used as the basis. This also corresponds to the encoded representation including the number 1 and the encoded representation including the number 2 in the preceding example.

FIG. 8 shows a schematic structural block diagram of an apparatus 800 for molecule encoding according to some embodiments in the disclosure. The apparatus 800 may be implemented or included in the electronic device 110, for example. The individual modules/components in the apparatus 800 may be implemented by hardware, software, firmware, or any combination thereof.

As shown in the figure, the apparatus 800 includes a root node determining module 801 configured to determine, based on a molecular fragment structure of a target molecule, a root node corresponding to the molecular fragment structure. A traversal record information determining module 802 configured to traverse the molecular fragment structure based on the root node to obtain traversal record information corresponding to the molecular fragment structure, the traversal record information corresponding to the molecular fragment structure indicating attribute information of each atom traversed along a traversal path corresponding to the traversal. An encoding result determining module 803 configured to determine, by performing encoding on the traversal record information corresponding to the molecular fragment structure using a specified encoding rule, an encoding result corresponding to the molecular fragment structure, the encoding result including an encoded representations of a plurality of atoms in the molecular fragment structure.

In some embodiments in the disclosure, the traversal record information determining module 802 may include: a traversal sub-module configured to traverse, based on a first traversal order, the atoms in the molecular fragment structure using the root node as a first node to be traversed; a traversal mark recording sub-module configured to record, for each traversed atom, a traversal mark of the atom, the traversal mark of each atom used to indicate at least a traversal path corresponding to the atom; an atom traversal record information generating sub-module configured to obtain, based on the traversal mark of each atom, traversal record information corresponding to each atom; and a molecule traversal record information generating sub-module configured to generate, based on the traversal record information corresponding to each atom, the traversal record information corresponding to the molecular fragment structure.

In some embodiments in the disclosure, the atom traversal record information generating sub-module is further configured to: determine, for the traversal mark of each traversed atom, in response to the traversal mark indicating that the atom is recorded in an existing traversal path, the atom as a ring atom; and generate the traversal record information corresponding to the atom, the traversal record information indicating at least a ring atom mark, a ring connection count, and a bond feature corresponding to the atom.

In some embodiments in the disclosure, the encoded representations of the plurality of atoms include strings, and the encoding result determining module 803 may be further configured to: perform encoding on the traversal record information corresponding to the molecular fragment structure using the specified encoding rule to obtain an initial encoding result, the initial encoding result including initial encoded representations of the plurality of atoms in the molecular fragment structure; and sort the initial encoded representations of the plurality of atoms according to a lexicographic order to obtain the encoding result corresponding to the molecular fragment structure.

In some embodiments in the disclosure, the encoded representations of the plurality of atoms include strings, and the encoding result determining module 803 may be further configured to: perform duplicate checking on the encoded representations of the plurality of atoms based on a second traversal order to obtain a duplicate checking result; perform deduplication on the repeated encoded atoms to obtain a deduplicated encoding result in response to the duplicate checking result indicating that there are repeated encoded atoms in the encoded representations of the plurality of atoms, the deduplicated encoding result including the encoded representations of the plurality of atoms retained after the deduplication; and sort the encoded representations of the plurality of atoms after the deduplication according to a lexicographic order to obtain the encoding result corresponding to the molecular fragment structure.

In some embodiments in the disclosure, the encoding result determining module 803 determines the deduplicated encoding result by: for each of the plurality of atoms retained after the deduplication, assigning a number the atom; updating the traversal record information corresponding to the molecular fragment structure based on the number; and performing encoding on the traversal record information corresponding to the molecular fragment structure to obtain the deduplicated encoding result, the deduplicated encoding result including the encoded representation of each of the plurality of atoms retained after the deduplication.

In some embodiments in the disclosure, the root node determining module 801 is configured to, in response to the molecular fragment structure including a first symmetric structure, the first symmetric structure indicating a symmetric structure centered on a central atom, designate the central atom of the first symmetric structure as a root node.

In some embodiments in the disclosure, the root node determining module 801 is configured to, in response to the molecular fragment structure including a second symmetric structure, the second symmetric structure indicating a symmetric structure centered on a bond, provide a virtual root node between pairs of atoms connected by the bond, and connect respectively the two atoms to the virtual root node. On this basis, the encoding result determining module 803 is further configured to delete an encoded representation corresponding to the virtual root node to obtain encoded representations of a plurality of remaining atoms, and sort the encoded representations of the plurality of remaining atoms to determine the encoding result corresponding to the molecular fragment structure.

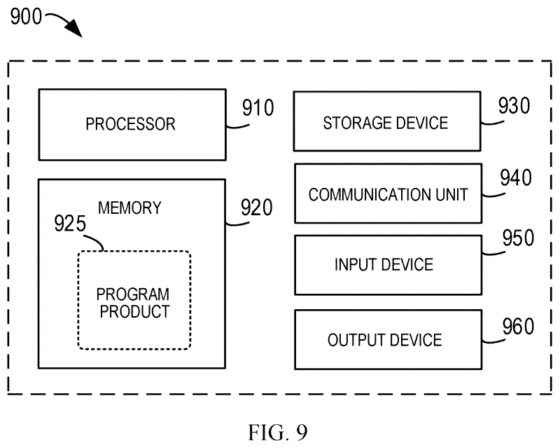

FIG. 9 shows a block diagram of an electronic device 900 in which one or more embodiments in the disclosure can be implemented. It should be understood that the electronic device 900 shown in FIG. 9 is only an example, and should not constitute any limitation to the function and scope of the embodiments described herein. The electronic device 900 shown in FIG. 9 may include or be implemented as the electronic device 110 in FIG. 1 or the apparatus 800 in FIG. 8.

As shown in FIG. 9, the electronic device 900 is in the form of a general-purpose electronic device. The components of the electronic device 900 may include, but are not limited to, one or more processors or processing units 910, a memory 920, a storage device 930, one or more communication units 940, one or more input devices 950, and one or more output devices 960. The processing unit 910 may be an actual or virtual processor and can perform various processing according to programs stored in the memory 920. In a multi-processor system, a plurality of processing units execute computer-executable instructions in parallel to improve the parallel processing capability of the electronic device 900.

The electronic device 900 usually includes a plurality of computer storage media. Such media may be any available media accessible by the electronic device 900, including but not limited to volatile and non-volatile media, and removable and non-removable media. The memory 920 may be a volatile memory (for example, a register, a cache, a random access memory (RAM)), a non-volatile memory (for example, a read-only memory (ROM), an electrically erasable programmable read-only memory (EEPROM), a flash memory), or a combination thereof. The storage device 930 may be a removable or non-removable medium, and may include a machine-readable medium, such as a flash drive, a magnetic disk or any other medium, which may be used to store information and/or data and may be accessed within the electronic device 900.

The electronic device 900 may further include additional removable/non-removable, volatile/non-volatile storage media. Although not shown in FIG. 9, a magnetic disk drive for reading or writing from a removable, non-volatile magnetic disk (for example, a “floppy disk”) and an optical disk drive for reading or writing from a removable, non-volatile optical disk may be provided. In these cases, each drive may be connected to a bus (not shown) by one or more data media interfaces. The memory 920 may include a computer program product 925 having one or more program modules configured to perform various methods or actions of various embodiments in the disclosure.

The communication unit 940 enables communication with other electronic devices through a communication medium. Additionally, the functions of the components of the electronic device 900 may be implemented in a single computing cluster or a plurality of computing machines that are capable of communicating through a communication connection. Therefore, the electronic device 900 may operate in a networked environment using logical connections to one or more other servers, network personal computers (PCs), or another network node.

The input device 950 may be one or more input devices, such as a mouse, a keyboard, a trackball, and the like. The output device 960 may be one or more output devices, such as a display, a speaker, a printer, and the like. The electronic device 900 may also communicate with one or more external devices (not shown) such as storage devices, display devices, etc. as needed through the communication unit 940, communicate with one or more devices that enable users to interact with the electronic device 900, or communicate with any device (for example, a network card, a modem, etc.) that enables the electronic device 900 to communicate with one or more other electronic devices. Such communication may be performed via an input/output (I/O) interface (not shown).

According to an example embodiment in the disclosure, a computer-readable storage medium is provided, on which computer-executable instructions are stored, and the computer-executable instructions are executed by a processor to implement the above-described method. According to an example embodiment in the disclosure, a computer program product is further provided, the computer program product is tangibly stored on a non-transitory computer-readable medium and includes computer-executable instructions, and the computer-executable instructions are executed by a processor to implement the above-described method.

According to an example embodiment in the disclosure, a computer program product or a computer program is provided. The computer program product or the computer program includes computer instructions, and the computer instructions are stored in a computer-readable storage medium. A processor of a computer device reads the computer instructions from the computer-readable storage medium, and the processor executes the computer instructions to cause the computer device to perform the method provided in various optional manners in FIG. 2. Therefore, details are not described herein again.

Various aspects in the disclosure are described herein with reference to flowcharts and/or block diagrams of the method, apparatus, device and computer program product implemented according to the disclosure. It should be understood that each block of the flowcharts and/or block diagrams and combinations of blocks in the flowcharts and/or block diagrams may be implemented by computer-readable program instructions.

These computer-readable program instructions may be provided to a processing unit of a general-purpose computer, a dedicated computer or other programmable data processing apparatus to produce a machine, so that the instructions, when executed by the processing unit of the computer or other programmable data processing apparatus, produce an apparatus for implementing functions/acts specified in one or more blocks of the flowcharts and/or block diagrams. These computer-readable program instructions may also be stored in a computer-readable storage medium. These instructions cause the computer, the programmable data processing apparatus and/or other devices to work in a specific manner, so that the computer-readable medium storing the instructions includes an article of manufacture, which includes instructions for implementing various aspects of the functions/acts specified in one or more blocks of the flowcharts and/or block diagrams.

The computer-readable program instructions may be loaded onto a computer, other programmable data processing apparatus, or other device to cause a series of operational steps to be performed on the computer, other programmable data processing apparatus or other device to produce a computer-implemented process, such that the instructions which execute on the computer, other programmable data processing apparatus or other device implement the functions/acts specified in one or more blocks in the flowcharts and/or block diagrams.

The flowcharts and block diagrams in the drawings illustrate the architecture, functionality, and operation of possible implementations of systems, methods and computer program products according to various embodiments in the disclosure. In this regard, each block in the flowcharts or block diagrams may represent a module, a program segment, or a portion of instructions, which includes one or more executable instructions for implementing specified logical functions. In some alternative embodiments, the functions noted in the blocks may also occur out of the order noted in the figures. For example, two blocks shown in succession may, in fact, be executed substantially concurrently, or the blocks may sometimes be executed in a reverse order, depending upon the functionality involved. It is also noted that each block of the block diagrams and/or flowcharts, and combinations of blocks in the block diagrams and/or flowcharts, may be implemented by special purpose hardware-based systems that perform the specified functions or acts, or combinations of special purpose hardware and computer instructions.

Various embodiments in the disclosure have been described above. The description is an example, not exhaustive, and is not limited to the disclosed embodiments. Many modifications and variations will be apparent to those of ordinary skill in the art without departing from the scope and spirit of the described embodiments. The terminology used herein was chosen to best explain the principles of the embodiments, the practical application or technical improvement over technologies in the market, or to enable others of ordinary skill in the art to understand the embodiments disclosed herein.

1. A method for molecule encoding, comprising:
determining, based on a molecular fragment structure of a target molecule, a root node corresponding to the molecular fragment structure;
traversing the molecular fragment structure based on the root node to obtain traversal record information corresponding to the molecular fragment structure, the traversal record information corresponding to the molecular fragment structure indicating attribute information of each atom traversed along a traversal path corresponding to the traversal; and
determining, by performing encoding on the traversal record information corresponding to the molecular fragment structure using a specified encoding rule, an encoding result corresponding to the molecular fragment structure, the encoding result comprising encoded representations of a plurality of atoms in the molecular fragment structure.

2. The method of claim 1, wherein traversing the molecular fragment structure to obtain the traversal record information corresponding to the molecular fragment structure comprises:
traversing, based on a first traversal order, the atoms in the molecular fragment structure using the root node as a first node to be traversed;
recording, for each traversed atom, a traversal mark of the atom, the traversal mark of each atom used to indicate at least a traversal path corresponding to the atom;
obtaining, based on the traversal mark of each atom, traversal record information corresponding to each atom; and
generating, based on the traversal record information corresponding to each atom, the traversal record information corresponding to the molecular fragment structure.

3. The method of claim 2, wherein obtaining, based on the traversal mark of each atom, the traversal record information corresponding to each atom comprises:
determining, for the traversal mark of each traversed atom, the atom as a ring atom in response to the traversal mark indicating that the atom is recorded in an existing traversal path; and
generating the traversal record information corresponding to the atom, the traversal record information indicating at least a ring atom mark, a ring connection count, and a bond feature corresponding to the atom.

4. The method of claim 1, wherein the encoded representations of the plurality of atoms comprise strings, and determining the encoding result corresponding to the molecular fragment structure comprises:
performing encoding on the traversal record information corresponding to the molecular fragment structure using the specified encoding rule to obtain an initial encoding result, the initial encoding result comprising initial encoded representations of the plurality of atoms in the molecular fragment structure; and
sorting the initial encoded representations of the plurality of atoms according to a lexicographic order to obtain the encoding result corresponding to the molecular fragment structure.

5. The method of claim 1, wherein the encoded representations of the plurality of atoms comprise strings, and determining the encoding result corresponding to the molecular fragment structure comprises:
performing duplicate checking on the encoded representations of the plurality of atoms based on a second traversal order to obtain a duplicate checking result;
performing deduplication on the repeated encoded atoms to obtain a deduplicated encoding result in response to the duplicate checking result indicating that there are repeated encoded atoms in the encoded representations of the plurality of atoms, the deduplicated encoding result comprising the encoded representations of the plurality of atoms retained after the deduplication; and
sorting the encoded representations of the plurality of atoms after the deduplication according to a lexicographic order to obtain the encoding result corresponding to the molecular fragment structure.

6. The method of claim 5, wherein the deduplicated encoding result is determined by:
for each of the plurality of atoms retained after the deduplication,
assigning a number to the atom;
updating the traversal record information corresponding to the molecular fragment structure based on the number; and
performing encoding on the traversal record information corresponding to the molecular fragment structure to obtain the deduplicated encoding result, the deduplicated encoding result comprising the encoded representation of each of the plurality of atoms retained after the deduplication.

7. The method of claim 1, wherein determining the root node corresponding to the molecular fragment structure comprises:
in response to the molecular fragment structure comprising a first symmetric structure and the first symmetric structure indicating a symmetric structure centered on a central atom, designating the central atom of the first symmetric structure as the root node.

8. The method of claim 1, wherein determining the root node corresponding to the molecular fragment structure comprises:
in response to the molecular fragment structure comprising a second symmetric structure and the second symmetric structure indicating a symmetric structure centered on a bond, providing a virtual root node between pairs of atoms connected by the bond, and connecting respectively the two atoms to the virtual root node; and
determining the encoding result corresponding to the molecular fragment structure comprises:
deleting an encoded representation corresponding to the virtual root node to obtain encoded representations of a plurality of remaining atoms; and
sorting the encoded representations of the plurality of remaining atoms to determine the encoding result corresponding to the molecular fragment structure.

9. An electronic device, comprising:
at least one processing unit; and
at least one memory coupled to the at least one processing unit and storing instructions executable by the at least one processing unit, the instructions, when executed by the at least one processing unit, cause the electronic device to perform operations comprising:
determining, based on a molecular fragment structure of a target molecule, a root node corresponding to the molecular fragment structure;
traversing the molecular fragment structure based on the root node to obtain traversal record information corresponding to the molecular fragment structure, the traversal record information corresponding to the molecular fragment structure indicating attribute information of each atom traversed along a traversal path corresponding to the traversal; and
determining, by performing encoding on the traversal record information corresponding to the molecular fragment structure using a specified encoding rule, an encoding result corresponding to the molecular fragment structure, the encoding result comprising encoded representations of a plurality of atoms in the molecular fragment structure.

10. The electronic device of claim 9, wherein traversing the molecular fragment structure to obtain the traversal record information corresponding to the molecular fragment structure comprises:
traversing, based on a first traversal order, the atoms in the molecular fragment structure using the root node as a first node to be traversed;
recording, for each traversed atom, a traversal mark of the atom, the traversal mark of each atom used to indicate at least a traversal path corresponding to the atom;
obtaining, based on the traversal mark of each atom, traversal record information corresponding to each atom; and
generating, based on the traversal record information corresponding to each atom, the traversal record information corresponding to the molecular fragment structure.

11. The electronic device of claim 10, wherein obtaining, based on the traversal mark of each atom, the traversal record information corresponding to each atom comprises:
determining, for the traversal mark of each traversed atom, the atom as a ring atom in response to the traversal mark indicating that the atom is recorded in an existing traversal path; and
generating the traversal record information corresponding to the atom, the traversal record information indicating at least a ring atom mark, a ring connection count, and a bond feature corresponding to the atom.

12. The electronic device of claim 9, wherein the encoded representations of the plurality of atoms comprise strings, and determining the encoding result corresponding to the molecular fragment structure comprises:
performing encoding on the traversal record information corresponding to the molecular fragment structure using the specified encoding rule to obtain an initial encoding result, the initial encoding result comprising initial encoded representations of the plurality of atoms in the molecular fragment structure; and
sorting the initial encoded representations of the plurality of atoms according to a lexicographic order to obtain the encoding result corresponding to the molecular fragment structure.

13. The electronic device of claim 9, wherein the encoded representations of the plurality of atoms comprise strings, and determining the encoding result corresponding to the molecular fragment structure comprises:
performing duplicate checking on the encoded representations of the plurality of atoms based on a second traversal order to obtain a duplicate checking result;
performing deduplication on the repeated encoded atoms to obtain a deduplicated encoding result in response to the duplicate checking result indicating that there are repeated encoded atoms in the encoded representations of the plurality of atoms, the deduplicated encoding result comprising the encoded representations of the plurality of atoms retained after the deduplication; and
sorting the encoded representations of the plurality of atoms after the deduplication according to a lexicographic order to obtain the encoding result corresponding to the molecular fragment structure.

14. The electronic device of claim 13, wherein the deduplicated encoding result is determined by:
for each of the plurality of atoms retained after the deduplication,
assigning a number to the atom;
updating the traversal record information corresponding to the molecular fragment structure based on the number; and
performing encoding on the traversal record information corresponding to the molecular fragment structure to obtain the deduplicated encoding result, the deduplicated encoding result comprising the encoded representation of each of the plurality of atoms retained after the deduplication.

15. The electronic device of claim 9, wherein determining the root node corresponding to the molecular fragment structure comprises:
in response to the molecular fragment structure comprising a first symmetric structure and the first symmetric structure indicating a symmetric structure centered on a central atom, designating the central atom of the first symmetric structure as the root node.

16. The electronic device of claim 9, wherein determining the root node corresponding to the molecular fragment structure comprises:
in response to the molecular fragment structure comprising a second symmetric structure and the second symmetric structure indicating a symmetric structure centered on a bond, providing a virtual root node between pairs of atoms connected by the bond, and connecting respectively the two atoms to the virtual root node; and
determining the encoding result corresponding to the molecular fragment structure comprises:
deleting an encoded representation corresponding to the virtual root node to obtain encoded representations of a plurality of remaining atoms; and
sorting the encoded representations of the plurality of remaining atoms to determine the encoding result corresponding to the molecular fragment structure.

17. A non-transitory computer-readable storage medium having a computer program stored thereon, the computer program being executable by a processor to perform operations comprising:
determining, based on a molecular fragment structure of a target molecule, a root node corresponding to the molecular fragment structure;
traversing the molecular fragment structure based on the root node to obtain traversal record information corresponding to the molecular fragment structure, the traversal record information corresponding to the molecular fragment structure indicating attribute information of each atom traversed along a traversal path corresponding to the traversal; and
determining, by performing encoding on the traversal record information corresponding to the molecular fragment structure using a specified encoding rule, an encoding result corresponding to the molecular fragment structure, the encoding result comprising encoded representations of a plurality of atoms in the molecular fragment structure.

18. The storage medium of claim 17, wherein traversing the molecular fragment structure to obtain the traversal record information corresponding to the molecular fragment structure comprises:
traversing, based on a first traversal order, the atoms in the molecular fragment structure using the root node as a first node to be traversed;
recording, for each traversed atom, a traversal mark of the atom, the traversal mark of each atom used to indicate at least a traversal path corresponding to the atom;
obtaining, based on the traversal mark of each atom, traversal record information corresponding to each atom; and
generating, based on the traversal record information corresponding to each atom, the traversal record information corresponding to the molecular fragment structure.

19. The storage medium of claim 18, wherein obtaining, based on the traversal mark of each atom, the traversal record information corresponding to each atom comprises:
determining, for the traversal mark of each traversed atom, the atom as a ring atom in response to the traversal mark indicating that the atom is recorded in an existing traversal path; and
generating the traversal record information corresponding to the atom, the traversal record information indicating at least a ring atom mark, a ring connection count, and a bond feature corresponding to the atom.

20. The storage medium of claim 17, wherein the encoded representations of the plurality of atoms comprise strings, and determining the encoding result corresponding to the molecular fragment structure comprises:
performing encoding on the traversal record information corresponding to the molecular fragment structure using the specified encoding rule to obtain an initial encoding result, the initial encoding result comprising initial encoded representations of the plurality of atoms in the molecular fragment structure; and
sorting the initial encoded representations of the plurality of atoms according to a lexicographic order to obtain the encoding result corresponding to the molecular fragment structure.
